SEQUENCE LISTING (14 sequences)

SEQ ID NO: 1 - DNA, 24 bp, Artificial Sequence
  synthetic oligonucleotide
cataggatcagatcagttgcgctc

SEQ ID NO: 2 - DNA, 33 bp, Artificial Sequence
  synthetic oligonucleotide
gcactagtgcaatcgcacttgaacgatgatctg

SEQ ID NO: 3 - DNA, 35 bp, Artificial Sequence
  synthetic oligonucleotide
cagcggccgctcataggatcagatcagttgcgctc

SEQ ID NO: 4 - DNA, 33 bp, Artificial Sequence; identical to SEQ ID NO: 2
  synthetic oligonucleotide

SEQ ID NO: 5 - DNA, 23 bp, Artificial Sequence
  synthetic oligonucleotide
ctcataggatcagatcagttgcg

SEQ ID NO: 6 - DNA, 21 bp, Artificial Sequence
  synthetic oligonucleotide
caactgatctgatcctatgag

SEQ ID NO: 7 - DNA, 19 bp, Artificial Sequence
  synthetic oligonucleotide
gcaatcgcacttgaacgat

SEQ ID NO: 8 - DNA, 23 bp, Artificial Sequence
  synthetic oligonucleotide
gatcatcgttcaagtgcgattgc

SEQ ID NO: 9 - DNA, 24 bp, Artificial Sequence
  synthetic oligonucleotide
cataggatcagatcagttgcgcnn

SEQ ID NO: 10 - DNA, 33 bp, Artificial Sequence
  synthetic oligonucleotide
gcactagtgcaatcgcacttgaacgatgatcnn

SEQ ID NO: 11 - DNA, 862 bp, Artificial Sequence
  bacterial ampicillin resistant gene
atgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaacagcggtaagatccttgagagttttcgccccgaagaacgttttccaatgatgagcacttttaaacttctgctatgtggcgcggtattatcccgtattgacgccgggcaagagcaactcggtcgccgcatacactattctcagaatgacttggttgagtactcaccactcacagaaaagcatcttacggatggcatgacagtaagagaattatgcagtgctgccataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagcgtgacaccacgatgcctgtagcaatggcaacaacgttgcgcaaactattaactggcgaactacttactctagcttcccggcaacaattaatagactggatggaggcggataaagttgcaggaccacttctgcgctcggccccttccggctggctggtttattgctgataaatctggagccggtgagcgtgggtctcgcggtatcattgcagcactggggccagatggtaagccctcccgtatcgtagttatctacacgacggggagtcaggcaactatggatgaacgaaatagacagatcgctgagataggtgcctcactgattaagcattggtaa

SEQ ID NO: 12 - DNA, 8 bp, Artificial Sequence
  synthetic DNA sequence
aaaaaaaa

SEQ ID NO: 13 - DNA, 7 bp, Artificial Sequence
  synthetic nucleotide sequence
nnnnnnn

SEQ ID NO: 14 - DNA, 7 bp, Artificial Sequence
  synthetic nucleotide sequence
nnnnnnn

(19) United States
(12) Patent Application Publication
(10) Pub. No.: US 2004/0259109 A1
(43) Pub. Date: Dec. 23, 2004

(54) Method of analyzing prokaryotic gene expression

(75) Inventors: Katsuhisa Suzuki, Kisarazu-shi (JP); Tsuneaki Watanabe, Kisarazu-shi (JP)

Correspondence Address: OBLON, SPIVAK, MCCLELLAND, MAIER & NEUSTADT, P.C., 1940 DUKE STREET, ALEXANDRIA, VA, 22314, US

(73) Assignee: AISIN SEIKI KABUSHIKI KAISHA, Kariya-shi (JP)

(21) Appl. No.: 10/724,837

(22) Filed: Dec. 2, 2003

(30) Foreign Application Priority Data
Dec. 2, 2002 (JP) 2002-350243

(51) Int. Cl. C12Q 1/68; C12P 19/34

(52) U.S. Cl. 435/6; 435/91.2

(57) ABSTRACT

A method of analyzing prokaryotic gene expression is disclosed, which comprises a process for isolating an mRNA, a process for adding a polyA at the 3′ end of the mRNA, a process for synthesizing a cDNA from the mRNA, a process for preparing a cDNA fragment having a sequence of a first adaptor at one end and a sequence of a second adaptor at the other end from the cDNA, a process for performing PCR with the cDNA fragment using a first primer having a sequence complementary to the sequence of the first adaptor and a second primer 2 having a sequence complementary to the sequence of the second adaptor, a process for performing electrophoresis with amplified cDNA fragments, and a process for recovering a desired cDNA based on the result of electrophoresis.

### FIELD OF THE INVENTION

The present invention relates to a method of analyzing gene expression, and particularly to a method of analyzing gene expression for a prokaryotic organism.

### BACKGROUND OF THE INVENTION

A conventional method of analyzing gene expression with respect to a eukaryotic organism is disclosed in, for example, International Publication No. WO 02/48352 A1. In this method, an mRNA is isolated from a eukaryotic cell, and a cDNA is synthesized from the mRNA. Thereafter, the cDNA is processed and then used in the analysis of gene expression.

However, in the case of analyzing the gene expression of a prokaryotic organism, since the prokaryotic mRNA does not have a polyA sequence, the analysis of gene expression has not yet been possible by the conventional methods.

### SUMMARY OF THE INVENTION

The object of the present invention is to provide a method of analyzing prokaryotic gene expression, which readily allows the analysis of gene expression for a prokaryotic organism.

In order to achieve the above object, the present invention provides a method of analyzing prokaryotic gene expression which comprises an mRNA isolation process for isolating an mRNA from a prokaryotic cell; a polyA addition process for adding a polyA to 3′ end of the mRNA; a cDNA synthesis process for synthesizing a cDNA from the polyA-added mRNA; a cDNA processing process for producing cDNA fragments attached with adaptors from the synthesized cDNA, having the sequence of a first adaptor (hereinafter referred to as Adaptor 1) at one end and the sequence of a second adaptor (hereinafter referred to as Adaptor 2) at the other end; a first PCR process for performing PCR (Polymerase Chain Reaction) with the cDNA fragment attached with adaptors using a first primer (hereinafter referred to as Primer 1) having a sequence complementary to the sequence of Adaptor 1 and a second primer (hereinafter referred to as Primer 2) having a sequence complementary to the sequence of Adaptor 2; an electrophoresis process for performing electrophoresis with a cDNA fragment amplified in the first PCR process; and a cDNA fragment recovery process for recovering a desired cDNA fragment based on the result of the electrophoresis process.

According to the present invention, an mRNA is first isolated from a prokaryotic cell. A polyA tail is then added to the 3′ end of the isolated mRNA, and using this polyA tail, a cDNA is synthesized from the mRNA. Subsequently, cDNA fragments attached with adaptors, having a sequence of Adaptor 1 at one end and a sequence of Adaptor 2 at the other end, are prepared from the synthesized cDNA in the cDNA processing process. Then, PCR is performed with these adaptor-attached cDNA fragments, using Primer 1 having a sequence complementary to the sequence of Adaptor 1 and Primer 2 having a sequence complementary to the sequence of Adaptor 2. Electrophoresis is then performed with the cDNA fragments amplified in the PCR, and a desired cDNA fragment is recovered based on the result of the electrophoresis to be used in the analysis of gene expression.

In such process, since a polyA is added to the mRNA isolated from a cell, the synthesis of cDNA can be carried out easily using the resultant mRNA. Thus, the analysis of gene expression can be carried out easily even for a prokaryotic organism.

Further, cDNA fragments can be amplified in large amounts because cDNA fragments having adaptor sequences at both ends of the cDNA are prepared and PCR is performed using a primer set having sequences complementary to these adaptor sequences. Thus, even if a desired cDNA fragment is present in a group of cDNA fragments in a low concentration, it can be amplified to a large extent and can be detected easily in electrophoresis. Furthermore, as described below, since it is possible to selectively amplify a portion of cDNA fragments from a group of cDNA fragments having adaptor sequences at both ends by selecting Primer 1 and Primer 2 appropriately, the analysis of gene expression may be carried out more easily.

Any adaptor-attached cDNA fragments may be used here as long as they have the sequence of Adaptor 1 at one end and the sequence of Adaptor 2 at the other end. That is, this group of cDNA fragments may be a group including virtually all of the genes expressed in a cell used in isolation, or a group including a portion of such expressed genes. Further, these cDNA fragments my be those in which adaptor sequences are added at the ends of whole-length cDNAs, or those in which adaptor sequences are added at the ends of a portion of the fragments of whole-length cDNAs. Furthermore, although the sequence of Adaptor 1 and the sequence of Adaptor 2 may respectively comprise any arbitrary base sequences, preferred are those that are designed in consideration of the efficiency etc. of the PCR to be performed later. That is to say, if each of these adaptor sequences has more or less 15 bases, it is possible to carry out stable PCRs such that cDNA fragments can be efficiently amplified.

Primer 1 may be any of those having a sequence complementary to the sequence of Adaptor 1. The complementary sequence as described here is not limited to the sequences that are 100% complementary to Adaptor 1, but also includes those sequences having a substantially complementary sequence that are sufficient to allow the amplification of cDNA fragments in the PCR. Further, Primer 1 is not limited to those comprising only the sequences complementary to the sequence of Adaptor 1, but may also include those having other sequences further connected to the sequences complementary to the sequence of Adaptor 1. Further, Primer 1 is not limited to those corresponding to the entire sequence of Adaptor 1 and may also include those corresponding to a portion of the sequence of Adaptor 1.

Likewise, Primer 2 may be any of those having a sequence complementary to the sequence of Adaptor 2. The complementary sequence as described herein is also not limited to the sequences that are 100% complementary to Adaptor 2, but also includes those substantially complementary sequence that are sufficient to allow the amplification of cDNA fragments in the PCR. Further, Primer 2 is not limited to those comprising only the sequences complementary to the sequence of Adaptor 2, but may also include those having other sequences further connected to the sequences complementary to the sequence of Adaptor 2. Further, Primer 2 is not limited to those corresponding to the entire sequence of Adaptor 2 and may also include those corresponding to a portion of the sequence of Adaptor 2.

For the primers, those having an arbitrary dibasic sequence NN at the 3′ end can be used. The term “arbitrary dibasic sequence NN” as used herein indicates the sequences arbitrarily selected from A, T, G and C. Dibasicity of each arbitrary sequence here is the result of considering the simplicity of the corresponding method and the accuracy of the analysis. For example, by making each arbitrary sequence dibasic, 256 types of primer sets can be obtained, and thus the group of cDNA fragments can be classified into 256 types of groups. As a result, one group may contain relatively fewer types of easily analyzable cDNA fragments. Further, it is also possible to make this arbitrary dibasic sequence NN to be of three or more bases for one primer on one side or both primers on both sides. Therefore, this increases the types of primers, and there can be 1024 types or 4096 types of primer sets.

Next, various reagents used generally in the PCRs will be explained below.

DNA polymerase is preferably not permanently inactivated even when heated at high temperatures for a short time upon denaturing the DNA chain in the PCR, and preferably has activity at high temperatures. For example, mention may be made of those DNA polymerases originating from thermostable bacteria, such as Thermococcus litoralis, Bacillus stearothermophilus, Methanothermus fervidus, Thermus aquaticus, T. flavus, T. lacteus, T. rubens and T. rubber; DNA polymerases originating from thermophilic archaea, such as Desulfurococcus mobilis, methanobacterium thermoautotrophilcum, Sulfolobus solfataricus, S. acidocaldarius, Thermoplasma acidophilum, and Pyrococcus kodakaraensis strain KOD1; and the like. Among these, for the reason of high availability, it is preferred to use the DNA polymerase originating from Thermus aquaticus (Taq DNA Polymerase), the DNA polymerase originating from Thermocuccus litoralis, or the DNA polymerase originating from Pyrococcus kodakaraensis strain KOD1.

In addition, an antibody specific to DNA polymerase may be mixed into the PCR reaction solution, in order to inhibit the activity of DNA polymerase prior to the amplification of nucleic acids. For this antibody, mention may be made of a monoclonal antibody, a polyclonal antibody, an antibody prepared from the recombinant technology, an antibody fragment prepared chemically or by the recombinant technology (e.g., Fab fragment). Among these, it is particularly preferred to use a monoclonal antibody. For example, a known monoclonal antibody for Taq DNA polymerase can inhibit the enzymatic activity of Taq DNA polymerase at about 20° C. to 40° C., but simultaneously it is inactivated by the high temperature of the thermal cycle of the PCR.

Further, the PCR is generally carried out in the presence of four types of dNTP, namely, dATP, dCTP, dGTP and dTTP.

The PCR is also generally carried out in a reaction solution containing a suitable buffering agent for the purpose of efficiently amplifying nucleic acids. The buffer solution can be appropriately modified, depending on the DNA polymerase or the like used in the reaction, in order to obtain the optimal conditions for the reaction. For example, a buffer solution in which potassium chloride or magnesium chloride is added to a Tris-type buffer solution with its pH appropriately adjusted can be used. Also, 5% to 10% of DMSO and 1% to 2% of betaine may be added to the PCR solution. This has an effect of minimizing the problem that the products are not easily amplified when the cDNA fragment acting as the template DNA has a secondary structure.

In the electrophoresis process, fractions of cDNA fragments can be obtained by electrophoresis of cDNA fragments (the product of the PCR), by means of any known planar gel electrophoresis such as acrylamide gel electrophoresis and agarose gel electrophoresis. Further, capillary column electrophoresis can be also used. For such electrophoresis, any known electrophoretic device may be used.

In the cDNA fragment recovery process, any process may be used as long as it recovers the desired cDNA fragment based on the result of electrophoresis.

For example, mention may be made of a process of performing electrophoresis by providing cDNA fragments to a DNA sequencer for detecting a marker substance, determining the cDNA fragment to be recovered from the results of an analysis thereof, subsequently performing electrophoresis with the same sample again, and when the desired cDNA fragment is detected, recovering the corresponding cDNA fragment by cutting out the portion of the gel containing the fragment.

Mention may also be made of a process in which after the gel electrophoresis, the entire gel is placed on a scanner for detecting a marker substance, the cDNA fragment to be recovered is determined from the results of an analysis thereof, and then the portion of the gel containing the desired fragment is cut out, from which the corresponding cDNA fragment is recovered.

Furthermore, in the above-described method of analyzing gene expression of a prokaryotic organism, the mRNA isolation process can have the process of isolating the whole RNA from the prokaryotic cell; the process of hybridizing a first nucleotide (hereinafter referred to as Nucleotide 1) having a sequence complementary to a portion of 16S rRNA with the 16S rRNA, and simultaneously hybridizing a second nucleotide (hereinafter referred to as Nucleotide 2) having a sequence complementary to a portion of 23S rRNA with the 23S rRNA; the process of hybridizing a first tag substance (hereinafter referred to as Tag Substance 1) to which is added a third nucleotide (hereinafter referred to as Nucleotide 3) having a sequence complementary to a site that is different from the site complementary to the 16S rRNA in Nucleotide 1, with the hybrid of the 16S rRNA and the Nucleotide 1, and simultaneously hybridizing a second tag substance (hereinafter referred to as Tag Substance 2) to which is added a fourth nucleotide (hereinafter referred to as Nucleotide 4) having a sequence complementary to a site that is different from the site complementary to the 23S rRNA in Nucleotide 2, with the hybrid of the 23S rRNA and the Nucleotide 2; and the process of removing the hybrid of 16S rRNA, Nucleotide 1 and Tag Substance 1 added with Nucleotide 3, and simultaneously removing the hybrid of 23S rRNA, Nucleotide 2 and Tag Substance 2 added with Nucleotide 4, from the whole RNA.

For a prokaryotic organism, since there is no polyA tail in the mRNA as described above, the process of purifying mRNA isolated from the whole RNA using the polyA tail cannot be employed.

In contrast, the process of mRNA isolation of the present invention involves first isolating the whole RNA from a prokaryotic cell. Then, with respect to the isolated whole RNA, Nucleotide 1 having a sequence complementary to a portion of 16S rRNA is. hybridized with 16S rRNA, and simultaneously Nucleotide 2 having a sequence complementary to a portion of 23S rRNA is hybridized with 23S rRNA. Subsequently, Tag Substance 1 to which is added Nucleotide 3 having a sequence complementary to a site that is different from the site complementary to 16S rRNA in Nucleotide 1, is hybridized with the hybrid of 16S rRNA and Nucleotide 1. Further, Tag Substance 2 to which is added Nucleotide 4 having a sequence complementary to a site that is different from the site complementary to 23S rRNA in Nucleotide 2, is hybridized with the hybrid of 23S rRNA and Nucleotide 2. After this, the hybrid of 16S rRNA with Nucleotide 1 and Tag Substance 1 added with Nucleotide 3 is removed, and simultaneously the hybrid of 23S rRNA with Nucleotide 2 and Tag Substance 2 added with Nucleotide 4 is removed, from the whole RNA.

According to this process, most of 16S rRNA and 23S rRNA that are abundantly present in the whole RNA can be removed from the whole RNA. Thus, mRNA can be purified more easily with higher purity.

Here, Nucleotide 1 can be any one as long as it has a sequence complementary to a portion of 16S rRNA and a sequence complementary to Nucleotide 3. Therefore, it may have a sequence other than the sequence complementary to a portion of 16S rRNA and the sequence complementary to Nucleotide 3. As used herein, the sequence complementary to a portion of 16S rRNA is not limited to the sequence which is 100% complementary to a portion of 16S rRNA but includes the sequences which are substantially complementary to the extent of being capable to be hybridized with 16S rRNA under suitable conditions. Likewise, the sequence complementary to Nucleotide 3 is not limited to the sequence which is 100% complementary to Nucleotide 3, but includes the sequences which are substantially complementary to the extent of being capable of hybridization with Nucleotide 3 under suitable conditions. Further, the sequence complementary to Nucleotide 3 in Nucleotide 1 is not limited to those corresponding to the entirety of Nucleotide 3 and may be those corresponding to a portion of Nucleotide 3.

Similarly, Nucleotide 2 can be any one as long as it has a sequence complementary to a portion of 23S rRNA and a sequence complementary to Nucleotide 4. Therefore, it may have a sequence other than the sequence complementary to a portion of 23S rRNA and the sequence complementary to Nucleotide 4. As used herein, the sequence complementary to a portion of 23S rRNA is also not limited to the sequence which is 100% complementary to a portion of 23S rRNA but includes the sequences which are substantially complementary to the extent of being capable to be hybridized with 23S rRNA under suitable conditions. Likewise, the sequence complementary to Nucleotide 4 is also not limited to the sequence which is 100% complementary to Nucleotide 4, but includes the sequences which are substantially complementary to the extent of being capable of hybridization with Nucleotide 4 under suitable conditions. Further, the sequence complementary to Nucleotide 4 in Nucleotide 2 is also not limited to those corresponding to the entirety of Nucleotide 4 and may be those corresponding to a portion of Nucleotide 4.

Meanwhile, Nucleotide 3 may be any one as long as it has a sequence complementary to a site separate from the site complementary to 16S rRNA in Nucleotide 1. Accordingly, it may have a sequence other than the sequence complementary to a portion of Nucleotide 1. As used herein, the sequence complementary to a portion of Nucleotide 1 is also not limited to the sequence which is 100% complementary to Nucleotide 1 as described above, but includes those sequences which are substantially complementary to the extent of being capable of hybridization with Nucleotide 1 under stable conditions.

Similarly, Nucleotide 4 may be any one as long as it has a sequence complementary to a site separate from the site complementary to 23S rRNA in Nucleotide 2. Therefore, it may have a sequence other than the sequence complementary to a portion of Nucleotide 2. As used herein, the sequence complementary to a portion of Nucleotide 2 is also not limited to the sequence which is 100% complementary to Nucleotide 2, as described above, but includes the sequences which are substantially complementary to the extent of being capable to be hybridized with Nucleotide 2 under suitable conditions.

For a tag substance, any substance may be used as long as the combination comprising the tag substance in the whole RNA could be removed using the properties of the substance. For example, mention may be made of magnetic beads. When this is used as a tag substance, magnetic beads can be precipitated using a magnetic stand or the like, so that only the combination comprising the tag substance can be precipitated and removed.

Furthermore, as the method for analyzing prokaryotic gene expression as described in above, there may be mentioned a method for analyzing prokaryotic gene expression wherein the Nucleotide 1 and the Nucleotide 2 are identical with each other in sequence, having sequences complementary to the sequence commonly possessed by the 16S rRNA and the 23S rRNA; Nucleotide 3 and Nucleotide 4 are also identical with each other; and the Tag Substance 1 and Tag Substance 2 are also identical with each other.

According to the present invention, Nucleotide 1 and Nucleotide 2 comprise the same sequence which has a sequence complementary to the sequence common to the 16S rRNA and 23S rRNA. Nucleotide 3 and Nucleotide 4 also comprise the same sequence. Tag Substance 1 and Tag Substance 2 are also the same substances.

Therefore, only one species would be sufficient for Nucleotide 1 and Nucleotide 2, for Nucleotide 3 and Nucleotide 4, and for Tag Substance 1 and Tag Substance 2, respectively. This makes the removal of 16S rRNA and 23S rRNA much easier.

Furthermore, in any one of the above-described methods for analyzing prokaryotic gene expression, the cDNA synthesis process may comprise synthesis of the cDNA and addition of a tag substance to the 5′ end of the cDNA at the same time, and the cDNA processing process may comprise a first cleavage process of cleaving the cDNA with type I restriction enzyme; the first recovery process of recovering the cDNA fragments having the above-mentioned tag substance by binding the cDNA fragments to a substance having high affinity to the tag substance; the Adaptor 1 binding process of binding the sequence of Adaptor 1 having a sequence complementary to the sequence of the cleavage site of the type I restriction enzyme, to the cDNA fragment having the tag substance; the second cleavage process of cleaving the cDNA fragments to which the sequence of Adaptor 1 is bonded, with type II restriction enzyme; the second recovery process of removing the cDNA fragments having no tag substance by binding the to the high-affinity substance, and recovering the cDNA fragments having no tag substance; and the Adaptor 2 binding process to binding the sequence of Adaptor 2 having a sequence complementary to the sequence of cleavage site of the type II restriction hydrogen, to the cDNA fragments having no tag substance.

According to the present invention, in the cDNA synthesis process, the synthesis of cDNA and addition of a tag substance at the 5′ end of the cDNA take place at the same time. In the cDNA processing process, cDNA is first cleaved with type I restriction enzyme. Then, by binding the fragments to a high-affinity substance having high affinity to the tag substance, the cDNA fragments having the tag substance are recovered, and the cDNA fragments not having the tab substance are removed. That is, the cDNA fragments at the 5′ end (polyT end) of cDNA are recovered, and the cDNA fragments at the 3′ end are removed. After this, to the recovered cDNA fragments, the sequence of Adaptor 1 having a sequence complementary to the sequence at the cleavage site of the type I restriction enzyme is bonded. Alternatively, after the cDNA is cleaved with type I restriction enzyme, to the cDNA fragments, the sequence of Adaptor 1 having a sequence complementary to the sequence at the cleavage site of the type I restriction enzyme is bonded. Subsequently, by binding a high-affinity substance having high affinity to the tag substance, the cDNA fragments having the tag substance are recovered, and the cDNA fragments not having the tag substance are removed.

Next, the cDNA fragments bonded with the sequence of Adaptor 1 are cleaved by the type II restriction enzyme. Subsequently, by binding the cDNA fragments to a high-affinity substance, at this time the cDNA fragments having the tag substance are removed, and the cDNA fragments not having the tag substance are recovered. That is, the cDNA fragments on the side bonded with Adaptor 1 are recovered, and the cDNA fragments on the side without Adaptor 1 are removed. To thus recovered cDNA fragments, the sequence of Adaptor 2 having a sequence complementary to the sequence at the cleavage site by the type II restriction enzyme, is bonded.

Or, after being cleaved by the type II restriction enzyme, the cDNA fragments are bonded with the sequence of Adaptor 2 having a sequence complementary to the sequence at the cleavage site by the type II restriction enzyme. Subsequently, by binding with a high-affinity substance, the cDNA fragments having the tag substance are removed, and the cDNA fragments not having the tag substance are recovered.

In this way, adaptor-attached cDNA fragments having the sequence of Adaptor 1 at one end and the sequence of Adaptor 2 at the other end can be easily prepared. Further, thus prepared group of cDNA fragments allows virtually all genes expressed in a cell, that is, known genes as well as unknown genes, to be included in the group. Accordingly, it is possible to utilize them effectively in the analysis of gene expression.

Furthermore, according to the present invention, the high-affinity substance used in the first recovery process may be combined with the tag substance during the first recovery process, but it is also possible, for example, to combine it with the tag substance, prior to the first cleavage process.

In addition, the high-affinity substance used in the second recovery process, may be also combined with the tag substance during the second recovery process, but it is also possible, for example, to combine it with the tag substance, prior to the second cleavage process.

Here, for the tag substance and the high-affinity substance, any substance may be used as long as it constitutes a bond capable of binding specifically with each other with high affinity.

In addition, a restriction enzyme is generally an enzyme which is also referred to as a restriction endonuclease, and is an enzyme which hydrolyzes and cuts two-stranded DNA at specific sequences. In the process as described above, two types of restriction enzymes (type I restriction enzyme and type II restriction enzyme) are combined and used to obtain appropriate fragments. It is preferred that the restriction enzyme used can cleave cDNA fragments into fragments with recognizable lengths. It is also preferred that the restriction enzyme cleaves a larger number, preferably virtually all of the synthesized cDNA fragments. Further, the restriction enzyme may be a 4-base recognizing enzyme or a 6-base recognizing enzyme, but for the reason described above, it is preferred to use a 4-base recognizing enzyme.

Furthermore, an adaptor sequence is used to bind the primer used during the PCR amplification and is designed to correspond to the restriction enzyme used. That is, the sequence of Adaptor 1 to be bound to the enzymatic cleavage site of type I restriction enzyme has a sequence complementary to the enzymatic cleavage site of type I restriction enzyme, while the sequence of Adaptor 2 to be bound to the enzymatic cleavage site of type II restriction enzyme has a sequence complementary to the enzymatic cleavage site of type II restriction enzyme.

As one of the above-described methods for analyzing prokaryotic gene expression, a method of analyzing prokaryotic gene expression may be used, wherein gel electrophoresis is performed in the electrophoresis process, and a portion of gel containing a desired cDNA fragment is cut out from the above-obtained gel, and then the corresponding cDNA fragment is recovered in the cDNA fragment recovery process.

According to the present invention, the electrophoresis process is carried out by means of gel electrophoresis. Also, for the cDNA fragment recovery process, a portion of gel containing a desired cDNA fragment is cut out from the gel with which electrophoresis has been carried out, and the cDNA fragment is recovered.

As a group of cDNA fragments is fractionated by gel electrophoresis in this manner, it is possible to improve the capability of size resolution, compared with, for example, capillary column electrophoresis. For this reason, it is possible to recover only the desired cDNA fragment more specifically.

Furthermore, as one of the above-described methods for analyzing prokaryotic gene expression, a method may be used wherein at least one of the above-described Primer 1 and Primer 2 is provided with a marker substance, and the marker substance is detected in the electrophoresis process.

According to the present invention, at least one of Primer 1 and Primer 2 is provided with a marker substance, and this marker substance is detected in the electrophoresis.

As such, when the PCR is carried out using the primers with a marker substance, the PCR products will also have the marker substance. Accordingly, even if the amount of the desired cDNA fragment is relatively small even after performing the PCR, the recognition of this marker substance during electrophoresis would make it possible to detect easily the position of the desired cDNA fragment on the gel.

For the marker substance, any one can be used as long as its detection sensitivity in electrophoresis is high. For example, fluorescent substances such as 6-carboxyfluorescein (hereinafter referred to as FAM), 4,7,2′,4′,5′,7′-hexachloro-6-carboxyfluorescein (hereinafter, referred to as HEX), NED (Applied Biosystems Japan, Ltd.) and 6-carboxy-X-rhodamine (hereinafter referred to as Rox) can be used. These marker substances may be bonded to, for example, an end of the primer DNA (for example, a 5′ end).

Furthermore, as one of the above-described methods for analyzing prokaryotic gene expression, the above-mentioned combination of the tag substance and the high-affinity substance may be any one of the combinations of biotin and streptavidin, of biatin and avidin, of FIGT and FITI antibody, and of DIG and anti-DIG.

As described in the above, any substance may be used for the tag substance and the high-affinity substance, if they constitute a binding pair which allows binding specifically with high-affinity. Among these, it is particularly preferred, from the perspectives of ease of handling, availability and the like, to use as the combination of the tag substance and the high-affinity substance as in the present invention, the combinations of biotin and streptavidin, of biatin and avidin, of FIGT and FITI antibody, and of DIG and anti-DIG. Further, in each of the combinations, any one of the two substances may be used as the tag substance and the other as the high-affinity substance.

Furthermore, as one of the above-described methods for analyzing prokaryotic gene expression, the ligation process of forming a recombinant plasmid, after the above-mentioned cDNA fragment recovery process, by ligating the recovered cDNA fragment to a plasmid vector, and the incorporation process of incorporating the recombinant plasmid into Escherichia coli may be provided.

According to the present invention, the ligation process is provided in which after the cDNA fragment recovery process, the recovered cDNA fragment is ligated to a plasmid vector to form a recombinant plasmid. Further, the incorporation process is provided in which the recombinant plasmid is incorporated into Escherichia coli.

As such, when the desired cDNA fragment is recovered and ligated to a plasmid vector and incorporated into E. coli, it will be useful for the case of interpreting the structure of the cDNA fragment or the like. That is, as the transformed E. coli is cultured, and a plasmid DNA with the cDNA fragment is isolated therefrom, this can be used in the structural analysis, for example, such as determination of the base sequences.

Furthermore, the method for analyzing prokaryotic gene expression as described above, may further comprise the second PCR process in which after the cDNA fragment recovery process and before the above-mentioned ligation process, PCR is carried out for the recovered cDNA fragment using a third primer (hereinafter referred to as Primer 3) having a sequence complementary to the sequence of Adaptor 1, and a fourth primer (hereinafter referred to as Primer 4) having a sequence complementary to the sequence of Adaptor 2.

According to the present invention, after the cDNA fragment recovery process and before the ligation process, the second PCR process is provided with respect to the recovered cDNA fragment, in which PCR is carried out using Primer 3 having a sequence complementary to the sequence of Adaptor 1, and Primer 4 having a sequence complementary to the sequence of Adaptor 2.

Upon carrying out such process, even when the amount of the cDNA fragment recovered from the cDNA fragment recovery process is small, the fragment can be amplified to a large extent. Thus, it is possible to ligate the cDNA fragment to a plasmid vector and to incorporate it into E. coli efficiently.

In addition, Primer 3 used in this PCR process may be one having a sequence complementary to the sequence of Adaptor 1, and for example, Primer 1 may also be used. However, in consideration of the incorporation process afterwards, if Primer 3 having a sequence for recognition of a suitable restriction enzyme is used, the incorporation process can be carried out effectively and certainly. Likewise, Primer 4 may be one having a sequence complementary to the sequence of Adaptor 2, and for example, Primer 2 may also be used. However, if Primer 4 having a sequence for recognition of the suitable restriction enzyme is used, the incorporation process can be carried out effectively and certainly.

### BRIEF DESCRIPTION OF THE DRAWINGS

FIG. 1 is an illustration showing a summary of a method of preparing a cDNA fragment group to be a sample for the recovery of a desired cDNA fragment.

FIG. 2 is an illustration showing the details of a method of preparing a cDNA fragment group to be the sample for the recovery of a desired cDNA fragment.

FIG. 3 is an illustration showing a method of removing rRNA.

FIG. 4(a) is an illustration showing a sequence of Adaptor 1.

FIG. 4(b) is an illustration showing the sequence of Adaptor 2.

FIG. 5 is an illustration showing the sequences of a primer set for the fractionation into 256 types of fragment groups.

FIG. 6 is an illustration showing a method of recovering a desired cDNA fragment from the cDNA fragment group.

FIG. 7 is an illustration showing the base sequence of an ampicillin-resistant gene, and especially of the Hha I cleavage site and Sau3A I cleavage site.

FIG. 8 is a diagram of photograph showing the electrophoresis results for a group of the cDNA fragment amplified by RT-PCR.

### DETAILED DESCRIPTION OF THE PRESENT INVENTION

Hereinafter, the Example of the present invention will be explained with reference to the drawings.

First, the genes expressed in a prokaryotic cell are classified as follows. That is, an entire RNA is isolated from a prokaryotic cell, and mRNA is further purified out. Then, to the 3′ end of the mRNA a polyA is added. This polyA is then used to synthesize a group of cDNAs from the mRNA. Then, the obtained groups of cDNAs are cleaved by two appropriate restriction enzymes and are bonded with adaptor sequences at both ends. Thus, groups of adaptor-attached cDNA fragments having a recognizable length and having adaptor sequences at both ends are prepared. Subsequently, the groups of adaptor-attached cDNA fragments are classified into 256 groups using 256 types of primer sets.

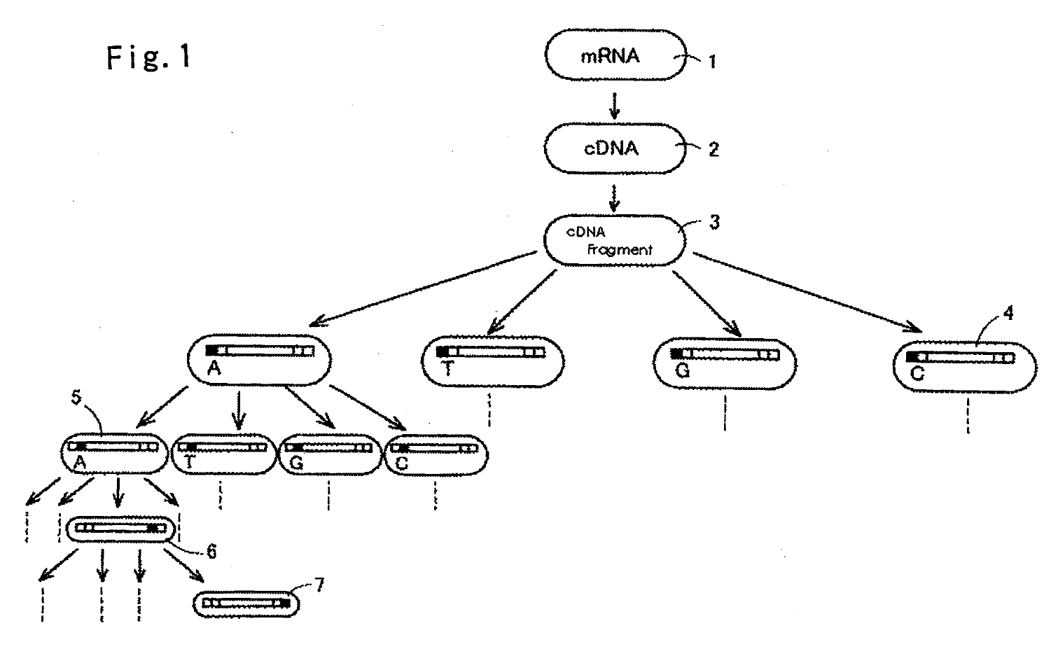

This technique for classification will be explained with reference to FIG. 1. From Group (1) comprising mRNAs, Group (2) of cDNA is synthesized. This is subjected to cleavage by two appropriate restriction enzymes to obtain Group (3) of cDNA fragments. Then, Group (3) of cDNA fragments is classified depending on the sequence which is tetrabasic in total, with dibasic sequences each at both ends of a cDNA fragment, that is, depending on how the four bases correspond to A, T, G and C. Specifically, the cDNA fragments are classified into four Groups (4) first according to the base at the 5′ end (shown as filled in black in FIG. 1). Then, these are again classified into 16 Groups (5) according to the second base next to the base at the end (shown as filled in black in FIG. 1). These are classified into 64 Groups (6) again according to the second base to the base at the 3′ end (shown as filled in black in FIG. 1), and classified again into 256 Groups (7) according to the base at the 3′ end (shown as filled in black in FIG. 1).

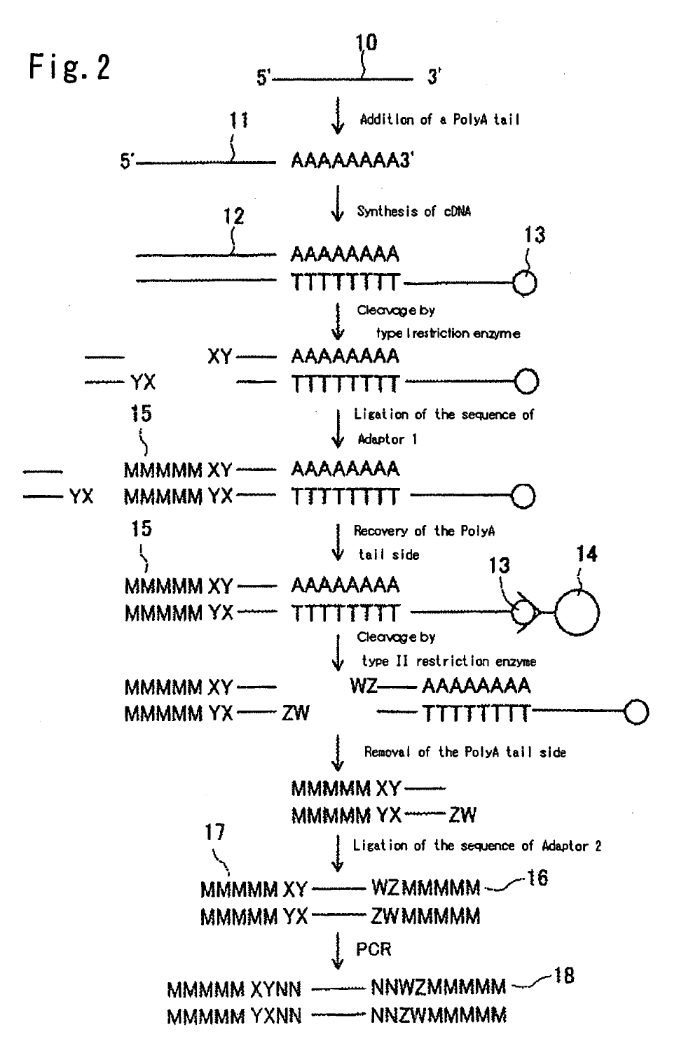

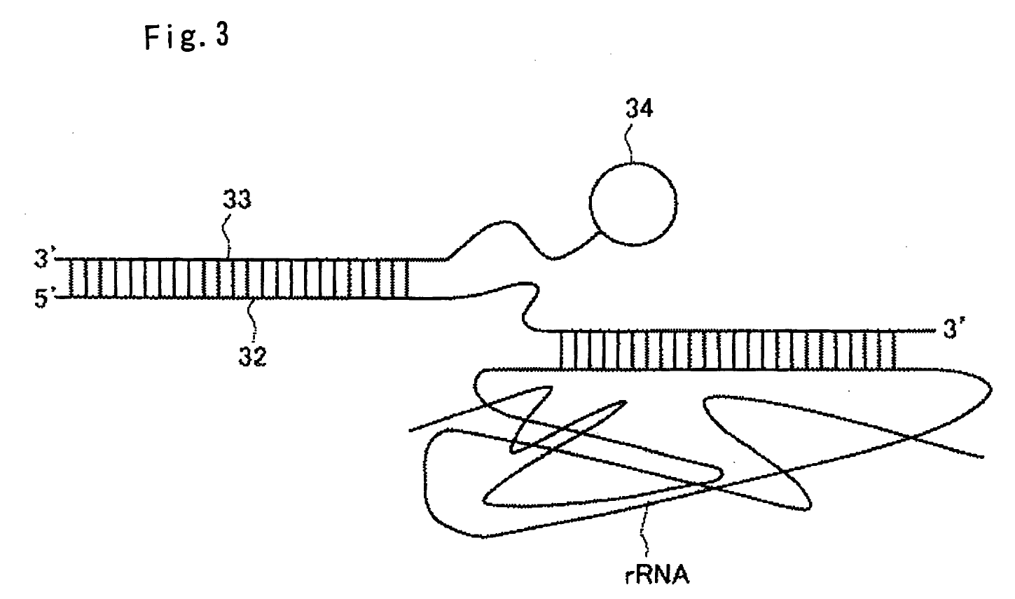

Next, a specific explanation will be given, with reference to FIG. 2 and FIG. 3, for a method of isolating mRNA from cells and preparing 256 types of cDNA fragment Group (7) from Group (1) of mRNA. The letters in FIG. 2 represent the bases constituting the base sequences, respectively, and N, N, W, X, Y and Z represent any optional bases, while X binds to Y and W binds to Z complementarily.

For the Example herein, in performing analysis of prokaryotic gene expression, the ampicillin-resistant gene (b1aM) that has been inserted into E. coli was taken as the target gene for carrying out an interpretation.

First, in the mRNA isolation process, mRNA (10) is isolated from the prokaryotic cells to be tested.

Specifically, the whole RNA is isolated from prokaryotic cells. Subsequently, Nucleotide 1 having a sequence complementary to a portion of 16S rRNA is hybridized with 16S rRNA, and simultaneously Nucleotide 2 having a sequence complementary to a portion of 23S rRNA is hybridized with 23S rRNA. Next, Tag Substance 1 to which is added Nucleotide 3 having a sequence complementary to a site that is different from the site complementary to 16S rRNA in Nucleotide 1, is hybridized with the combination of 16S rRNA and Nucleotide 1. Further, Tag Substance 2 to which is added Nucleotide 4 having a sequence complementary to a site that is different from the site complementary to 23S rRNA in Nucleotide 2, is hybridized with the combination of 23S rRNA and Nucleotide 2. Subsequently, from the whole RNA, the hybrid of 16S rRNA, Nucleotide 1 and Tag Substance 1 added with Nucleotide 3 is removed, and simultaneously the hybrid of 23S rRNA, Nucleotide 2 and Tag Substance 2 added with Nucleotide 4 is removed.

Preferably, Nucleotide 1 and Nucleotide 2 are identical ones having a sequence complementary to a common sequence present in both 16S rRNA and 23S rRNA. Nucleotide 3 and Nucleotide 4 are also identical ones. Likewise, Tag Substance 1 and Tag Substance 2 are also identical ones.

Thus, as shown in FIG. 3, after the whole RNA is isolated, Nucleotide 1 (32) having a sequence complementary to the common sequence present in both 16S rRNA and 23S rRNA, is hybridized with either 16S rRNA or 23S rRNA. Next, Tag Substance 1 (34) to which is added Nucleotide 3 (33) having a sequence complementary to a site that is different from the site complementary to 16S rRNA in Nucleotide 1, is hybridized with the respective hybrid of 16S rRNA or 23S rRNA and Nucleotide 1 (32). Then, the respective hybrid of 16S rRNA or 23S rRNA and Tag Substance 1 (34) added with Nucleotide 1 (32) and Nucleotide 3 (33), is removed from the whole RNA.

In the present Example, Escherichia coli having an ampicillin-resistant gene, i.e., E. coli (DH5α strain) having the plasmid pBluescript was cultured, and the whole RNA was first isolated from the cells. This isolation of the whole RNA was carried out using an RNA isolation kit (Rneasy Protect Bacterial Mini Kit), according to the appended manual. Subsequently, mRNA was purified using 10 μg of the isolated whole RNA. MICROB Express Bacterial mRNA Isolation Kit (product of Ambion) was used for this purification. The purification procedure was carried out in accordance with the appended manual.

Next, in the polyA addition process, a polyA was added to the 3′ end of mRNA (10) to obtain mRNA (11) added with the polyA (See FIG. 2).

The reaction composition for the polyA addition reaction is as follows. The composition for the addition of PolyA Polymerase (product of Takara Shuzo Co., Ltd.) was referred to for the preparation of 5×polyA buffer, and PolyA Polymerase used was a product of Takara Shuzo Co., Ltd.

| mRNA 500 ng | X | μl |
|---|---|---|
| 5 × polyA buffer | 4 | μl |
| 5 × MnCl2 | 4 | μl |
| PolyA Polymerase | 1 | μl |
| DEPC-treated water | 11 − X | μl |
| Total | 20 | μl |

The above reaction solution was incubated at 37° C. for 1 hour. Subsequently, 180 μl of TE buffer and 200 μl of a phenol-chloroform solution was added to the reaction solution and mixed. Then, this mixture was centrifuged at 15,000 rpm for 5 minutes at room temperature. 185 μl (92.5 μl×2) of the supernatant liquid was taken. To this supernatant liquid, 18.5 μl of a 3 M sodium acetate solution was added and mixed. To this solution, 520 μl of 99.5% ethanol was added and mixed. Then, the solution was refrigerated at −80° C. for 10 minutes. This refrigerated solution was centrifuged at 4° C. for 5 minutes at 15,000 rpm, and the precipitate was rinsed with 70% ethanol. Subsequently, the rinsed precipitate was centrifuged at 15,000 rpm for 5 minutes at 4° C., and the supernatant liquid was removed. Subsequently, the precipitate was dried in the air and then dissolved in 7 μl of DEPC-treated water. Thus, a polyA tail was added to the 3′ end of mRNA.

Next, in the cDNA synthesis process, cDNA (12) is synthesized from the mRNA added with the polyA (11) using a reverse transcriptase. At the same time, a tag substance is added to the 5′ end of the cDNA. In the present Example, cDNA was synthesized using a primer in which an oligo-dT primer complementary to the polyA tail at the 3′ end of the mRNA was marked by biotin (a tag substance).

| | |
|---|---|
| PolyA mRNA solution | 7 μl |
| (the amount of mRNA was 500 ng) | |
| 10 mM dNTP Mix | 1 μl |
| 50 μM Biotin-Not I anchored dt18 | 2 μl |
| Total | 10 μl |

First, the above mixture was warmed at 65° C. for 5 minutes, and was left in an ice bath for at least 1 minute.

On the one hand, the following mixture was prepared. In preparation of the mixture, the Super Script First Strand System of Invitrogen Japan K.K. was used.

| | |
|---|---|
| 10 × RT buffer | 2 μl |
| 25 mM MgCl₂ | 4 μl |
| 0.1 M DTT | 2 μl |
| RNase OUT | 1 μl |
| Super Script II | 1 μl |
| Total | 10 μl |

This mixture was added to and mixed with the above-mentioned mixture refrigerated in an ice bath, and then was incubated at 42° C. for 1 hour. This reaction solution was taken as the 1^{st} Strand Mix.

On the other hand, the following mixture (this is referred to as the 2^{nd} Strand Mix) was prepared.

The mixtures used were the products of Invitrogen Japan K.K.

| | |
|---|---|
| 5 × 2^{nd} Strand buffer | 30 μl |
| 10 mM dNTP Mix | 4 μl |
| 0.1 M DTT | 2 μl |
| E. coli Ligase | 2 μl |
| E. coli Polymerase | 4 μl |
| RNase H | 1 μl |
| DEPC-treated water | 87 μl |
| Total | 130 μl |

This mixture was stored in an ice bath until the synthetic reaction for the above-mentioned 1^{st} strand was completed.

Next, 130 μl of the cooled 2^{nd} Strand Mix was added to and mixed with the 1^{st} Strand Mix, and then the mixture was reacted at 16° C. for 2 hours. It was further reacted at 70° C. for 15 minutes to inactivate the enzymes.

After completion of the reaction, 150 μl of TE buffer was added, and to this mixture, 300 μl of a phenol-chloroform solution (product of Wako Pure Chemical Co., Ltd.) was added and mixed. The mixture was centrifuged at 15,000 rpm for 5 minutes at room temperature. Subsequently, 280 μl of the supernatant liquid was taken, and to this 28 μl of a 3 M sodium acetate solution was added and mixed.

Subsequently, to the mixture, 770 μl of 99.5% ethanol was added and mixed, and the mixture was refrigerated at −80° C. for 10 minutes. Next, it was centrifuged at 15,000 rpm for 15 minutes at 4° C., and the precipitate was rinsed with 10% ethanol. The rinsed precipitate was centrifuged at 15,000 rpm for 3 minutes at 4° C., and the supernatant was removed. Then, the precipitate was dried in the air and was dissolved in 25 μl of 10 mM Tris. Thus, cDNA was synthesized from mRNA.

Next, the cDNA processing process will be described.

First, in the first cleavage process, thus synthesized cDNA (12) is cleaved by type I restriction enzyme.

In the present Example, the following mixture was prepared and left overnight at 37° C. Hha I, which is one of the 4-base recognition restriction enzymes, was used as type I restriction enzyme.

| | |
|---|---|
| cDNA solution | 23 μl |
| M buffer | 5 μl |
| Hha I (20 U) | 2 μl |
| Water | 20 μl |
| Total | 50 μl |

Next, the mixture was warmed at 65° C. for 15 minutes to inactivate the enzyme. Subsequently, to the mixture, 5 μl of a 3 M sodium acetate solution (product of Wako Pure Chemicals Co., Ltd.) was added, and to this mixture, 160 μl of 99.5% ethanol was added and mixed. The mixture was refrigerated at −80° C. for 10 minutes. Next, it was centrifuged at 15,000 rpm for 15 minutes at 4° C., and then the precipitate was rinsed with 70% ethanol. This precipitate was centrifuged at 15,000 rpm for 3 minutes at 4° C., and then the supernatant was removed. Subsequently, the precipitate was dried in the air.

Next, in the Adaptor 1 binding process, to the cleaved cDNA fragment, the sequence of Adaptor 1 having a sequence complementary to the sequence at the cleavage site of type I restriction enzyme is bonded.

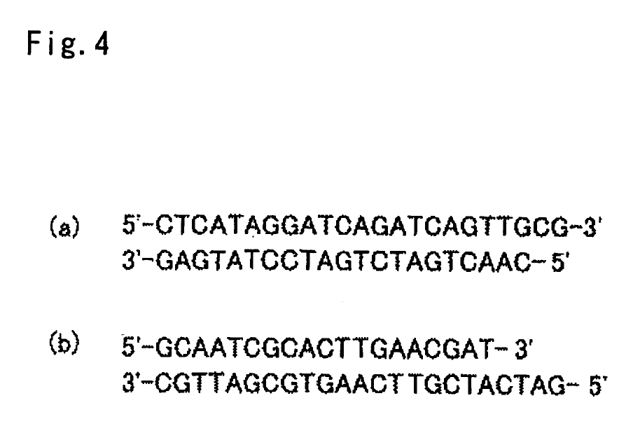

In the present Example, the precipitate dried in the air was dissolved in 5 μl of a 1 pmol/μl solution of Adaptor 1, 5 μl of Liquid I of the Ligation kit from Takara Shuzo Co., Ltd. was added to and mixed with the solution, which was reacted at 16° C. for 2 hours. The sequence of Adaptor 1 is shown in FIG. 4(a).

Next, in the first recovery process, a high-affinity substance having high-affinity is bonded with the tag substance, thereby recovering the cDNA fragment having the tag substance.

In the present Example, biotin (13) was captured using streptavidin (a high-affinity substance) (14), and only the 3′ end of the cleaved cDNA fragment was recovered. Specifically, 90 μl of TE buffer was added to the reaction solution, and to this mixture, 100 μl of a bead suspension (product of Dynal Biotech) was also added. By binding biotin with streptavidin (14) fixed onto the magnetic beads, the 3′ and of the cleaved cDNA fragment was recovered.

Next, in the second cleavage process, the recovered cDNA fragment was cleaved using type II restriction enzyme.

In the present Example, the following mixture (for 2 samples) was prepared. As the type I restriction enzyme, Sau3A I, which is one of the 4-base recognizing restriction enzyme, was used.

| | |
|---|---|
| Sau3A I | 10 μl |
| H buffer | 40 μl |
| Water | 250 μl |
| Total | 300 μl |

Then, 150 μl of the mixture solution was added to a tube containing the beads and mixed. The mixture is kept warm at 37° C. for 3 hours.

Next, in the second recovery process, the cDNA fragment having the tag substance is removed by binding with a high-affinity substance, and the cDNA fragment not having a tag substance is recovered.

Specifically, after completion of the above-mentioned reaction, the tube was stood on a magnetic stand to draw the magnetic beads, and 185 μl of the supernatant was transferred to a new tube. Then, 100 μl of TE buffer was again added to the tube containing the beads and mixed. Subsequently, the tube was stood on the magnetic stand again to draw the beads, and 85 μl of the supernatant was transferred to a new tube.

Next, to 270 μl of the sample, 27 μl of a 3 M sodium acetate solution, 750 μl of ethanol and 2 μl of paint pellet (Takara Shuzo Co., Ltd.) were added and mixed. The mixture was left overnight at −20° C. This mixture was centrifuged at 15,000 rpm for 15 minutes at 4° C., and then the precipitate was rinsed with 500 μl of 70% ethanol. This precipitate was centrifuged at 15,000 rpm for 3 minutes at 4° C., and then the supernatant was removed. The precipitate was dried in the air.

Next, in the Adaptor 2 binding process, the recovered cDNA fragment is bonded with the sequence of Adaptor 2 having a sequence complementary to the cleavage site of type II restriction enzyme.

In the present Example, the precipitate dried in the air was dissolved in 5 μl of 1 pmol/μl of Adaptor 2 solution, 5 μl of Solution I of the Ligation Kit version 2 of Takara Shuzo Co., Ltd. was added and mixed, and the mixture was then reacted at 16° C. for 2 hours. Further, the sequence of Adaptor 2 is shown in FIG. 4(b).

Next, 190 μl of TE buffer was added, and 200 μl of a phenol-chloroform solution was added and mixed. The solution as centrifuged at 15,000 rpm for 10 minutes at room temperature. Then, 185 μl of the supernatant was transferred to a new tube. To this, 20 μl of a 3 M sodium acetate solution was added, and 510 μl of 99.5% ethanol was added and mixed.

Subsequently, the mixture was refrigerated at −80° C. for 20 minutes. Next, this was centrifuged at 15,000 rpm for 15 minutes at 4° C., and then the precipitate was rinsed with 200 μl of 70% ethanol. The rinsed precipitate was centrifuged at 15,000 rpm for 3 minutes at 4° C., and then the supernatant was removed. Then, the precipitate was dried in the air and dissolved in 50 μl of TE buffer.

In the cDNA processing process as described above, a group of adaptor-attached cDNA fragments (17) comprising known sequences at both ends is obtained.

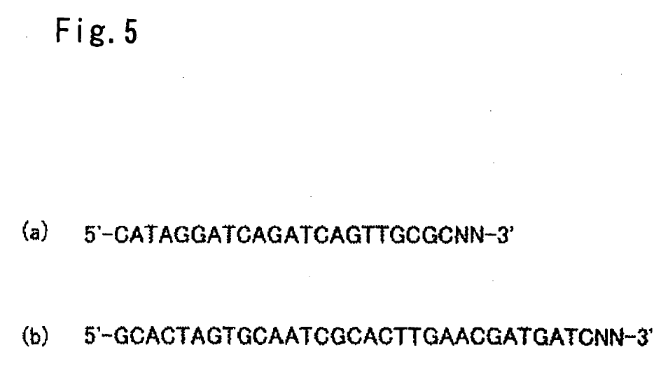

Next, in the first PCR process, with respect to the group of the adaptor-attached cDNA fragments (17), Primer 1 having a sequence complementary to the sequence of Adaptor 1, and Primer 2 having a sequence complementary to the sequence of Adaptor 2 are used in carrying out the PCR. In the present Example, as this primer set, use is made of a primer having a sequence complementary to the sequence of Adaptor 1 and an additional dibasic sequence in the direction of amplification (shown as NN in FIG. 5) as shown in FIG. 5(a), and a primer having a sequence complementary to the sequence of Adaptor 2 and an additional dibasic sequence in the direction of amplification (shown as NN in FIG. 5) as shown in FIG. 5(b). Since the dibase provided in the direction of amplification of each primer is designed from all of the possible combinations comprising the four types of bases, namely, A, T; G and C, 256 types of primer sets in total are envisaged. Therefore, by performing PCR for the group of cDNA fragments (17) using all of these primer sets, classification into 256 types of groups of cDNA fragments (18) and amplification in the PCR can be achieved at the same time. Further, the PCR may be carried out by any known means. 256 types of groups of cDNA fragments (18) thus obtained can be used as the sample for recovering the desired cDNA fragment.

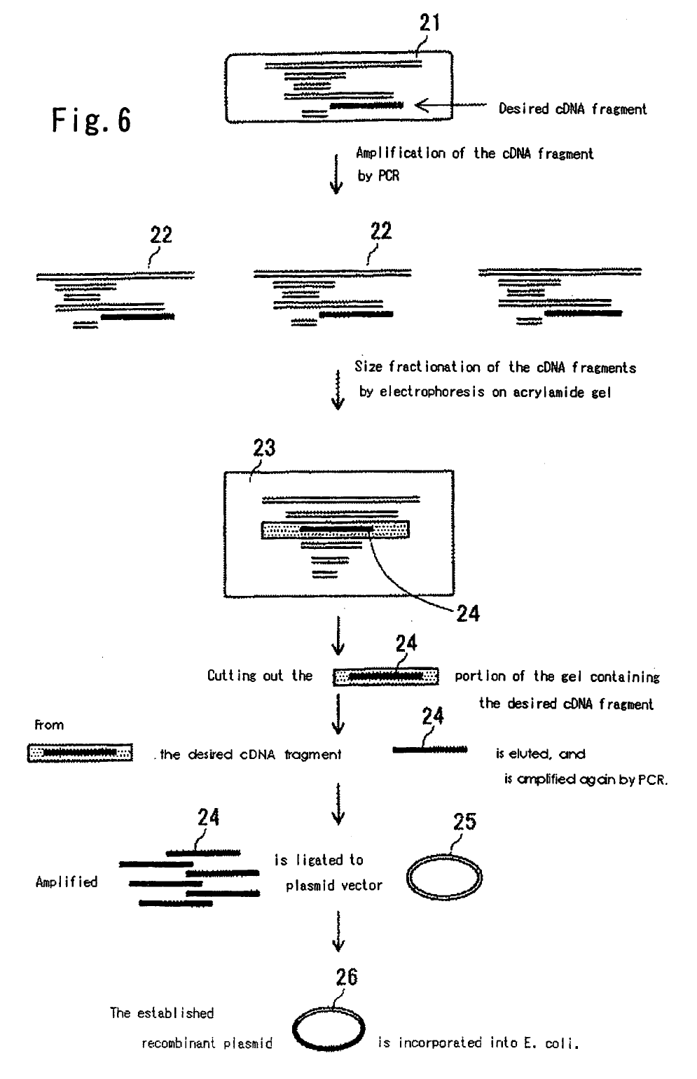

Next, one group of cDNA among the 256 types of groups of cDNA fragments is selected as a sample, and the desired cDNA fragment is recovered by the following means. The means for recovery will be described with reference to FIG. 6.

First, in the first PCR process, a group of cDNA fragments (21) are amplified by means the PCR using Primer 1 which has a sequence complementary to the sequence of Adaptor 1 (15) and is bonded with a marker substance, and Primer 2 which has a sequence complementary to the sequence of Adaptor 2 (16).

Specifically, Primer 1 in which a near-IR fluorescent dye IRD-800 as a fluorescent substance is bonded to Oligonucleotide (1) having a sequence complementary to the sequence of Adaptor 1 (15), and Primer 2 which comprises Oligonucleotide (2) having a sequence complementary to the sequence of Adaptor 2 (16) were used in performing the PCR. These Primer 1 and Primer 2 may be synthesized respectively by any known means.

Oligonucleotide (1):
5′-cataggatcagatcagttgcgctc-3′
Oligonucleotide (2):
5′-gcactagtgcaatcgcacttgaacgatgatctg-3′

The reaction composition of the PCR is as follows. For reference, KOD Dash, dNTPs, 10×buffer used were the products of Toyobo Co., Ltd.

| | |
|---|---|
| cDNA | 0.5 to 5 ng |
| KOD Dash | 2.5 U |
| dNTPs | 0.2 mM (the final concentration) |
| 10 × buffer | 5 μl |
| IRD-800 fluorescent- | 5 pmol |
| labeled primer | |
| Reverse primer | 10 pmol |
| Total | 50 μl |

Further, the PCR was carried out using GeneAmp 2400 (product of Perkin Elmer Co., Ltd.). For the conditions of the PCR, reference was made to the conditions of the Stepdown PCR (Biotechniques, 1996, 20:478-485).

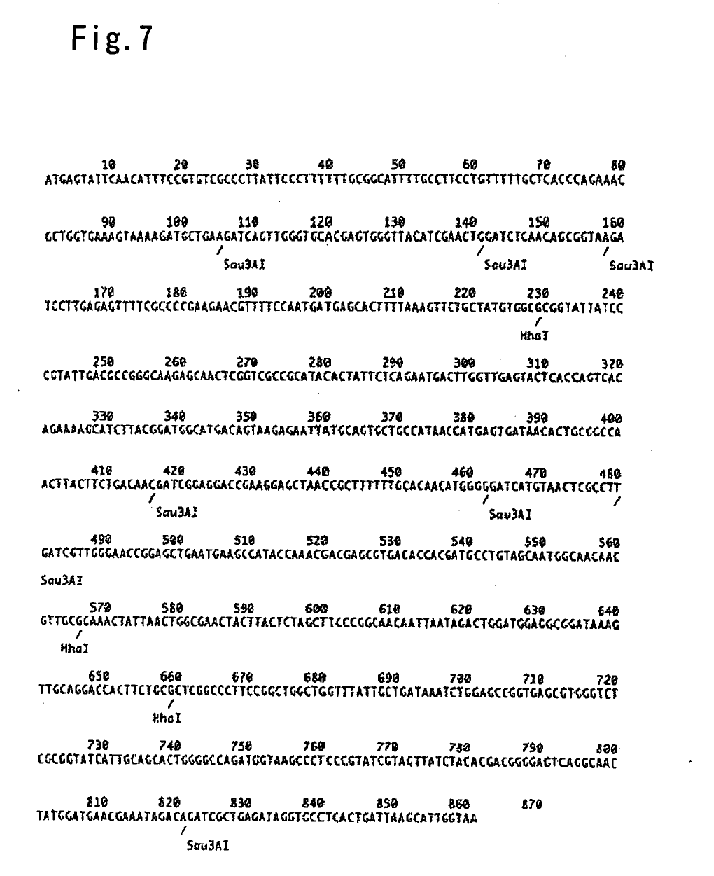

In the present Example, since the 3′ end of the ampicillin-resistant gene would be caught up, the 658-826 fragment of the ampilicin-resistant gene shown by the base sequence and the Hha I site and the Sau3A I site in FIG. 7 will be amplified. On considering the sequences of Adaptor 1 and Adaptor 2, the adaptor-attached cDNA fragment of 214 bp will be amplified.

Next, in the electrophoresis process, gel electrophoresis is carried out for the group of the cDNA fragments amplified in the first PCR process (22) (the PCR product).

In the present Example, acrylamide gel electrophoresis was performed for size exclusion by providing the group of amplified cDNA fragments (22) to a DNA sequencer (product of LI-COR, LIC-4200L(S)-1) which is capable of detecting a fluorescent dye. The composition of the acrylamide gel (23) followed the manual for the DNA sequencer.

Next, in the cDNA fragment recovery process, based on the results of electrophoresis, the portion containing the desired cDNA fragment (24) is cut out from the gel to recover the corresponding cDNA fragment (24).

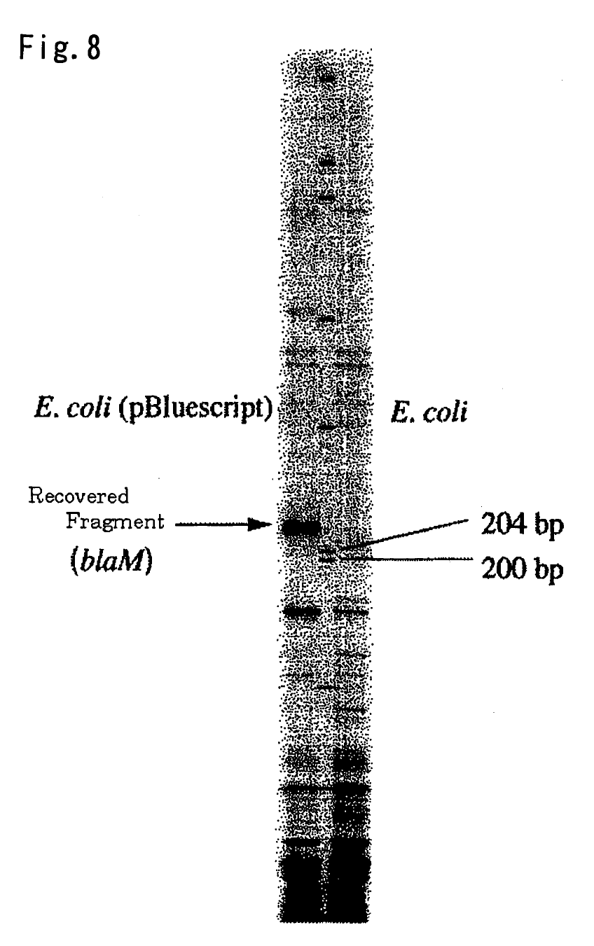

In the present Example, as a result of performing electrophoresis on acrylamide gel, a band (214 bp) appeared at the position as expected, as shown in FIG. 8. FIG. 8 is an image obtained from the sequencer data. Next, the gel on which electrophoresis was carried out was peeled off from the gel plate using filter paper. Then, the gel adhered to the filter paper was placed on a scanner which can detect the fluorescent dye, and the entire gel was imaged. Then, the site of the desired cDNA fragment on the gel was found, and the gel including the desired cDNA fragment was cut out. Furthermore, the gel of Odyssey imaging system (product of LI-COR Company) was used in the present Example. Subsequently, the desired cDNA fragment was recovered from the cut acrylamide gel. This isolation of DNA was done using E.Z.N.A. Poly-Gel DNA Extraction Kit of Omega Bio-tek Co.

Next, in the second PCR process, PCR is performed again with the recovered cDNA fragment (24) using Primer 3 having a sequence complementary to the sequence of Adaptor 1 (15) and Primer 4 having a sequence complementary to the sequence of Adaptor 2 (16). Specifically, PCR was carried out using Primer 3 in the forward, which has a sequence complementary to the sequence of Adaptor 1 as well as the sequence of the restriction enzyme (Not I) site, and using Primer 4 in the reverse, which has a sequence complementary to the sequence of Adaptor 2 as well as the sequence of the restriction enzyme (Spe I) site. Primer 3 comprises Oligonucleotide (3), and Primer 4 comprises Oligonucleotide (4).

Oligonucleotide (3):
5′-cagcggccgctcataggatcagatcagttgcgctc-3′
Oligonucleotide (4):
5′-gcactagtgcaatcgcacttgaacgatgatctg-3′

Further, the PCR was carried out also using GeneAmp 2400 (product of Perkin Elmer Co., Ltd.). For the conditions of the PCR, reference was made to the conditions of the Stepdown PCR (Biotechniques, 1996, 20:478-485). The used enzyme (DNA polymerase) was KOD Dash enzyme manufactured by Toyobo Co. The composition of the reaction solution was prepared according to the appended manual.

3′ end

Next, in the ligation process, the product of cDNA fragment (24) amplified in the second PCR process is ligated to a plasmid vector (25) to form a recombinant plasmid (26).

Specifically, the amplified product of cDNA fragment (24) was treated with restriction enzymes (Not I) and (Spe I), and then was ligated to a plasmid vector (pbluescript II) (25) to form a recombinant plasmid (26). This ligation was carried out according to the appended manual using Ligation Kit ver. 2 (product of Takara Shuzo Co., Ltd.).

Next, in the incorporation process, the recombinant plasmid (26) is incorporated into E. coli.

In the present Example, the recombinant plasmid (26) was incorporated into E. coli by a known technique, using E. coli DH5α as a competent cell.

Next, the recombinant plasmid was isolated form the transformed E. coli by a known technique. This isolation of plasmid was carried out with respect to a plurality of colonies. For each isolated recombinant plasmid, the base sequence of the inserted cDNA fragment was determined. As a result, it was confirmed that the cDNA fragment was the portion of the ampicillin-resistant gene.

As described above, in the present Example, mRNA is isolated from prokaryotic cells. Then, a polyA tail is added to the 3′ end of the isolated mRNA, and using this polyA tail, cDNA is synthesized form the mRNA. Next, in the cDNA processing process, prepared from the cDNA is an adaptor-attached cDNA fragment having the sequence of Adaptor 1 at one end and the sequence of Adaptor 2 at the other end. Then, with this adaptor-attached cDNA fragment, PCR is carried performed using Primer 1 having a sequence complementary to the sequence of Adaptor 1, and Primer 2 having a sequence complementary to the sequence of Adaptor 2. Subsequently, electrophoresis is performed on the cDNA fragment amplified in the PCR. Based on the results of this electrophoresis, the desired cDNA fragment is recovered and used in the analysis of gene expression.

In such a method, since a polyA is added to the mRNA isolated from cells, cDNA can be easily synthesized therefrom. Thus, even in the case of prokaryotic cells, analysis of gene expression thereof can be easily carried out.

Further, since PCR is carried out by preparing cDNA fragments having adaptor sequences at both ends from cDNA and using a primer set having sequences complementary to these adaptor sequences, cDNA fragments can be amplified in large amounts. Thus, even when the desired cDNA fragment present in the group of cDNA fragments is in a low concentration, this fragment can be amplified to a large extent and can be easily detected in electrophoresis. As described below, analysis of gene expression can also be easily carried out, because it is possible to amplify only a portion of cDNA fragments selectively among the group of cDNA fragments having adaptor sequences at both ends by appropriately selecting Primer 1 and Primer 2.

Furthermore, in the mRNA isolation process of the present Example, the whole RNA is isolated from prokaryotic cells. For the whole isolated RNA, Nucleotide 1 having a sequence complementary to a portion of 16S rRNA is hybridized with 16S rRNA, and simultaneously Nucleotide 2 having a sequence complementary to a portion of 23S rRNA is hybridized with 23S rRNA.

Then, Tag Substance 1 to which is added Nucleotide 3 having a sequence complementary to a site that is different from the site complementary to 16S rRNA in Nucleotide 1, is hybridized with the hybrid of 16S rRNA and Nucleotide 1. Further, Tag Substance 2 to which is added Nucleotide 4 having a sequence complementary to a site that is different from the site complementary to 23S rRNA in Nucleotide 2, is hybridized with the hybrid of 23S rRNA and Nucleotide 2. Subsequently, from the whole RNA, the hybrid of Tag Substance 1 to which is added 16S rRNA, Nucleotide 1 and Nucleotide 3 is removed, and simultaneously the hybrid of Tag Substance 2 to which is added 23S rRNA, Nucleotide 2 and Nucleotide 4 is removed.

More specifically, Nucleotide 1 and Nucleotide 2 comprise the same sequence having a sequence complementary as the common sequence present in both 16S rRNA and 23S rRNA. Nucleotide 3 and Nucleotide 4 also comprise the same sequence. Further, Tag Substance 1 and Tag Substance 2 are also identical.

Therefore, virtually all of 16S rRNA and 23S rRNA present in a large amount in the whole RNA can be removed from the whole RNA. Thus, mRNA can be easily purified to higher purity. Since it is sufficient with only one species each for Nucleotide 1 and Nucleotide 2, Nucleotide 3 and Nucleotide 4, and Tag Substance 1 and Tag Substance 2, removal of 16S rRNA and 23S rRNA can be carried out more easily.

Furthermore, in the present Example, synthesis of cDNA and addition of a tag substance at the 5′ end of cDNA are carried out at the same time in the cDNA synthesis process.

In the cDNA processing process, cDNA is first cleaved by type I restriction enzyme. Then, the sequence of Adaptor 1 having a sequence complementary to the sequence at the cleavage site of the type I restriction enzyme is bonded to the cleaved cDNA fragments. Then, by binding a high-affinity substance having high affinity to the tag substance, the cDNA fragments having the tag substance are recovered, and the cDNA fragments having no tag substance are removed. That is, the cDNA fragments at the 5′ end (polyT end) of cDNA are recovered, and the cDNA fragments at the 3′ end are removed.

Next, the cDNA fragments bonded with the sequence of Adaptor 1 is cleaved with type II restriction enzyme.

Subsequently, by binding with a high-affinity substance, the cDNA fragments having the tag substance are removed, and the cDNA fragments having no tag substance are recovered. That is, the cDNA fragments at the side bonded with Adaptor 1 are recovered, and the cDNA fragments at the side without Adaptor 1 are removed. Then, to the recovered cDNA fragments, the sequence of Adaptor 2 having a sequence complementary to the sequence at the cleavage site of the type II restriction enzyme is bonded.

For this reason, the adaptor-added cDNA fragments having the sequence of Adaptor 1 at one end and the sequence of Adaptor 2 at the other end can be easily prepared. Further, it is possible for the group of thus prepared cDNA fragments to include almost all of the genes expressed in the cell, namely, known genes and unknown genes equally. Therefore, the group can be effectively utilized in the analysis of gene expression.

Furthermore, in the present Example, gel electrophoresis is performed for the electrophoresis process. The cDNA fragment recovery process is carried out by cutting out the portion of gel containing the desired cDNA fragment from the gel on which electrophoresis has been performed, and by recovering the cDNA fragments.

If the group of cDNA fragments is fractionated by gel electrophoresis in this manner, it is possible to improve the capability of size resolution compared with, for example, capillary column electrophoresis. For this reason, only the desired cDNA fragments can be recovered more specifically.

Furthermore, in the present Example, Primer 1 is provided with a marker substance, and this marker substance is detected in electrophoresis.

As such, when PCR is carried out using a primer having a marker substance, the PCR product will also have the marker substance. Therefore, even if the amount of the desired cDNA fragment is relatively small even after performing the PCR, it is possible to detect easily the position of the desired cDNA fragment in the gel by recognizing this marker substance in electrophoresis.

In the present Example, Hha I is used as type I restriction enzyme, and Sau3A I is used as type II restriction enzyme.

By using these enzymes, the cDNA fragments can be cleaved into fragments having recognizable lengths. Further, an even larger number of the synthesized cDNA fragments can be cleaved.

Furthermore, in the present Example, biotin is used as the tag substance and streptavidin is used as the high-affinity substance. It is particularly preferred to use these for the reasons such as the ease of handling or availability.

In the present Example, the cDNA fragment recovery process is followed by the ligation process in which the recovered cDNA fragment is ligated to a plasmid vector to form a recombinant plasmid. The incorporation process for incorporating the recombinant plasmid into E. coli is also provided.

As such, when the desired cDNA fragment is recovered and ligated to a plasmid vector and incorporated into E. coli, it becomes useful in the structural analysis of a cDNA fragment or the like. That is, as the transformed E. coli is cultured and the plasmid DNA having the cDNA fragment is isolated therefrom, this can be used in, for example, the structural analysis such as determination of base sequences.

Furthermore, in the present Example, there is provided, after the cDNA fragment recovery process and before the ligation process, the second PCR process in which PCR is performed with the recovered cDNA fragment using Primer 3 having a sequence complementary to the sequence of Adaptor 1 and Primer 4 having a sequence complementary to the sequence of Adaptor 2.

By performing such process, the cDNA fragment recovered from the cDNA fragment recovery process can be amplified to a large extent, even if the amount of the cDNA fragment is small. Thus, it is possible to ligate the cDNA fragment to a plasmid vector efficiently and to incorporate it into E. coli.

In the description given above, the embodiment of the present invention is described by means of Example; however, the present invention is not limited to the above Example but definitely it can be appropriately modified and applied within a scope of not deviating from the gist.

For example, in the cDNA processing process of the above-described Example, although for the first cleavage process, the first recovery process and the Adaptor 1 binding process, the process is performed in the order of the first cleavage process, the Adaptor 1 binding process and the first recovery process, the process may also be performed in the order of the first cleavage process, the first recovery process and the Adaptor 1 binding process.

In addition, in the cDNA processing process of the above Example, although the second cleavage process, the second recovery process and the Adaptor 2 binding process, the process is performed in the order of the second cleavage process, the second recovery process and the Adaptor 2 binding process, the process may also be performed in the order of the second cleavage process, the Adaptor 2 binding process and the second recovery process.

What is claimed is:

1. A method of analyzing prokaryotic gene expression comprising the processes of:
an mRNA isolation process for isolating an mRNA from a prokaryotic cell;
a polyA addition process for adding a polyA at the 3′ end of the mRNA;
a cDNA synthesis process for synthesizing a cDNA from the polyA-added mRNA;
a cDNA processing process for preparing an adaptor-attached cDNA fragment having the sequence of a first adapter at one end and the sequence of a second adapter at the other end, from the cDNA;
a first PCR process for performing PCR with the adaptor-attached cDNA fragments, using a first primer having a sequence complementary to the sequence of the first adaptor and a second primer having a sequence complementary to the sequence of the second adaptor;
an electrophoresis process for performing electrophoresis with the cDNA fragments amplified in the first PCR process; and
a cDNA fragment recovery process for recovering the desired cDNA fragment based on the result of the electrophoresis.

2. A method of analyzing prokaryotic gene expression according to claim 1, wherein the mRNA isolation process comprises:
a process of isolating the whole RNA from the prokaryotic cell;
a process of hybridizing a first nucleotide having a sequence complementary to a portion of 16S rRNA with the 16S rRNA, and simultaneously hybridizing a second nucleotide having a sequence complementary to a portion of 23S rRNA with the 23S rRNA;
a process of hybridizing a first tag substance Tag Substance 1) to which is added a third nucleotide having a sequence complementary to a site that is different from the site complementary to the 16S rRNA in the first nucleotide, with the hybrid of the 16S rRNA and the the first nucleotide, and simultaneously hybridizing a second tag substance to which is added a fourth nucleotide having a sequence complementary to a site that is different from the site complementary to the 23S rRNA in the second nucleotide, with the hybrid of the 23S rRNA and the second nucleotide; and
a process of removing the hybrid of the 16S rRNA, the first nucleotide and the first tag substance added with the third nucleotide, and simultaneously removing the hybrid of the 23S rRNA, the second nucleotide and the second tag substance added with the fourth nucleotide, from the whole RNA.

3. A method of analyzing prokaryotic gene expression according to claim 2, wherein
the first nucleotide and the second nucleotide are identical ones having a sequence complementary to the common sequence present in both 16S rRNA and 23S rRNA,
the third nucleotide and the fourth nucleotide are also identical, and
the first tag substance and the second tag substance are also identical.

4. A method of analyzing prokaryotic gene expression according to claim 1, wherein
the cDNA synthesis process comprises the synthesis of the cDNA as well as the addition of a tag substance at the 5′ end of the cDNA at the same time; and
the cDNA processing process comprises:
a first cleavage process for cleaving the cDNA by means of a type I restriction enzyme;
a first recovery process for recovering the cDNA fragments having the tag substance by binding a high-affinity substance having high affinity to the tag substance;
a binding process of the first adaptor for binding to the cDNA fragments having the tag substance, the sequence of the first adaptor having a sequence complementary to the sequence at the cleavage site of the type I restriction enzyme;
a second cleavage process for cleaving the cDNA fragment bonded with the sequence of the first adaptor by means of a type II restriction enzyme;
a second recovery process for removing the cDNA fragments with the tag substance and recovering the cDNA fragment not having the tag substance, by binding them with the high-affinity substance; and
a binding process of the second adaptor for binding to the cDNA fragment not having the tag substance, the sequence of a second adaptor having a sequence complementary to the sequence at the cleavage site of the type II restriction enzyme.

5. A method of analyzing prokaryotic gene expression according to claim 1, wherein
the electrophoresis process is carried out by means of gel electrophoresis; and
the cDNA fragment recovery process is carried out by cutting out the portion of gel containing the desired cDNA fragment from the gel and recovering the corresponding cDNA fragment.

6. A method of analyzing prokaryotic gene expression according to claim 1, wherein at least one part of the first primer and the second primer is(are) given with a marker substance, and the marker substance is detected in the electrophoresis.

7. A method of analyzing prokaryotic gene expression according to claim 4, wherein the combination of the tag substance and the high-affinity substance is any one of the combinations of biotin and streptavidin, of biatin and avidin, of FIGT and FITI antibody, and of DIG and anti-DIG.

8. A method of analyzing prokaryotic gene expression according to claim 1, which comprises, after the cDNA fragment recovery process,
a ligation process for ligating the recovered cDNA fragment to a plasmid vector to form a recombinant plasmid; and
an incorporation process for incorporating the recombinant plasmid into Escherichia coli.

9. A method of analyzing prokaryotic gene expression according to claim 8, which comprises, after the cDNA fragment recovery process and before the ligation process, a second PCR process for performing PCR with the recovered cDNA fragment, using a third primer having a sequence complementary to the sequence of the first adaptor and a fourth primer having a sequence complementary to the sequence of the second adaptor.
